(19) United States
(12) Patent Application Publication
(10) Pub. No.: US 2025/0132001 A1
(43) Pub. Date: Apr. 24, 2025

(54) LEARNING DEVICE, ACTION RECOMMENDATION DEVICE, LEARNING METHOD, ACTION RECOMMENDATION METHOD, AND STORAGE MEDIUM

(71) Applicant: NEC Corporation, Minato-ku, Tokyo (JP)

(72) Inventor: Ryosuke TOGAWA, Tokyo (JP)

(73) Assignee: NEC Corporation, Minato-ku, Tokyo (JP)

(21) Appl. No.: 18/692,894

(22) PCT Filed: Sep. 28, 2021
(86) PCT No.: PCT/JP2021/035571

§ 371 Date: Mar. 18, 2024

(51) Int. Cl. G16H 20/00; G16H 50/20; G16H 50/30

(52) U.S. Cl. CPC G16H 20/00; G16H 50/20; G16H 50/30

(57) ABSTRACT

An acquisition means 15X of a learning device 1X acquires history information indicating a history of a health state of a target person and an action of the target person contributing to variation in the health state, and success/failure information indicating whether or not the action contributed to the variation in the health state of the target person. Then, a learning means 16X of the learning device 1X trains a model through machine learning based on the history information and the success/failure information, wherein the model is configured to output information regarding a recommended action recommended to improve the health state of the target person upon receiving an input of the history information indicating the history of the action and the health state of the target person. It allows for determination of an optimized recommended action. “It can be used to support user's decision making.”

### TECHNICAL FIELD

The present disclosure relates to the art of a learning device, an action recommendation device, a learning method, an action recommendation device, and a storage medium for processing recommendations of actions for altering a health state of a target person.

### BACKGROUND

There are known devices or systems that suggest the action which a target person should take. For example, Patent Literature 1 discloses a system that collects basic information, body information, and action information of the user whose health state is regarded to be improved, and analyzes conditions under which a certain action contributes to improving the health state, thereby presenting a successful experience to the user in accordance with the physical function and the life habit of the user. Further, Patent Literature 2 discloses a system for providing the target person with an improved action portfolio that encourages action modification for health state improvement or the like based on data of the health diagnosis result and the like.

### CITATION LIST

### Patent Literature

Patent Literature 1: JP2015-200969A
Patent Literature 2: JP2009-157837A

### SUMMARY

### Problem to be Solved

When suggesting the action modification for health management of a target person, the appropriate actions to be taken next depends on the past actions and health state of the target person. On the other hand, Patent Literature 1 and Patent Literature 2 do not disclose a point of determining an action to be recommended in consideration of both the past actions and the health state of the target person.

In view of the above-described issue, it is therefore an example object of the present disclosure to provide a learning device, an action recommendation device, a learning method, an action recommendation method, and a storage medium capable of suitably determining an action to be recommended to a target person.

### Means for Solving the Problem

One aspect of the learning device is a learning device including:
an acquisition means configured to acquire history information indicating a history of a health state of a target person and an action of the target person contributing to variation in the health state, and success/failure information indicating whether or not the action contributed to the variation in the health state of the target person; and
a learning means configured to train a model based on the history information and the success/failure information, wherein the model is configured to output information regarding a recommended action recommended to improve the health state of the target person upon receiving an input of the history information indicating the history of the action and the health state of the target person.

One aspect of the action recommendation device is an action recommendation device including:
a history information acquisition means configured to acquire history information indicating a history of a health state of a target person and an action of the target person contributing to variation in the health state;
a recommended action determination means configured to determine a recommended action to be recommended to the target person based on the history information and a recommendation model; and
an output means configured to output information regarding the recommended action,
wherein the recommendation model is a model which learned a relation between each health state of subjects and each recommended action to be recommended to improve the each health state of the subjects, based on history information indicating histories of health states of the subjects and actions of the subjects which contribute to variation in the health states.

One aspect of the learning method is a learning method executed by a computer, the learning method including:
acquiring history information indicating a history of a health state of a target person and an action of the target person contributing to variation in the health state, and success/failure information indicating whether or not the action contributed to the variation in the health state of the target person; and
training a model based on the history information and the success/failure information, wherein the model is configured to output information regarding a recommended action recommended to improve the health state of the target person upon receiving an input of the history information indicating the history of the action and the health state of the target person.

One aspect of the action recommendation method is an action recommendation method executed by a computer, the action recommendation method including:
acquiring history information indicating a history of a health state of a target person and an action of the target person contributing to variation in the health state;
determining a recommended action to be recommended to the target person based on the history information and a recommendation model; and
outputting information regarding the recommended action,
wherein the recommendation model is a model which learned a relation between each health state of subjects and each recommended action to be recommended to improve the each health state of the subjects, based on history information indicating histories of health states of the subjects and actions of the subjects which contribute to variation in the health states.

One aspect of the storage medium is a storage medium storing a program executed by a computer, the program causing the computer to:
acquire history information indicating a history of a health state of a target person and an action of the target person contributing to variation in the health state, and success/failure information indicating whether or not the action contributed to the variation in the health state of the target person; and
train a model based on the history information and the success/failure information, wherein the model is configured to output information regarding a recommended action recommended to improve the health state of the target person upon receiving an input of the history information indicating the history of the action and the health state of the target person.

Another aspect of the storage medium is a storage medium storing a program executed by a computer, the program causing the computer to:
acquiring history information indicating a history of a health state of a target person and an action of the target person contributing to variation in the health state;
determining a recommended action to be recommended to the target person based on the history information and a recommendation model; and
outputting information regarding the recommended action,
wherein the recommendation model is a model which learned a relation between each health state of subjects and each recommended action to be recommended to improve the each health state of the subjects, based on history information indicating histories of health states of the subjects and actions of the subjects which contribute to variation in the health states.

### Effect

An example advantage according to the present disclosure is to suitably determine a recommended action for improving the health of a target person in consideration of the past action and health state of the target person.

### BRIEF DESCRIPTION OF THE DRAWINGS

FIG. 1 It illustrates a schematic configuration of an action recommendation system according to a first example embodiment.

FIG. 2A illustrates the hardware configuration of a learning device.

FIG. 2B illustrates the hardware configuration of an action recommendation device.

FIG. 3 It schematically illustrates the behavior of generating the recommendation model according to the SAIL method.

FIG. 4 It schematically illustrates the behavior of optimizing an action imitation engine.

FIG. 5 It is an example of functional blocks of the learning device.

FIG. 6 It schematically illustrates the learning of the recommendation model and the calculation of the recommended action using the recommendation model.

FIG. 7 It is an example of a flowchart illustrating a learning process of a recommendation model to be executed by the learning device.

FIG. 8 It is an example of a functional block diagram of the action recommendation device.

FIG. 9 It is an example of a flowchart of an action recommendation process executed by an action recommendation device.

FIG. 10 It illustrates a schematic configuration of an action recommendation system according to a second example embodiment.

FIG. 11 It is a block diagram of the learning device in a third example embodiment.

FIG. 12 It is an example of a flowchart executed by the learning device in the third example embodiment.

FIG. 13 It is a block diagram of an action recommendation device in a fourth example embodiment.

FIG. 14 It is an example of a flowchart executed by the action recommendation device in the fourth example embodiment.

### EXAMPLE EMBODIMENTS

Hereinafter, example embodiments of a learning device, an action recommendation device, a learning method, an action recommendation method, and a storage medium will be described with reference to the drawings.

### First Example Embodiment

### (1) System Configuration

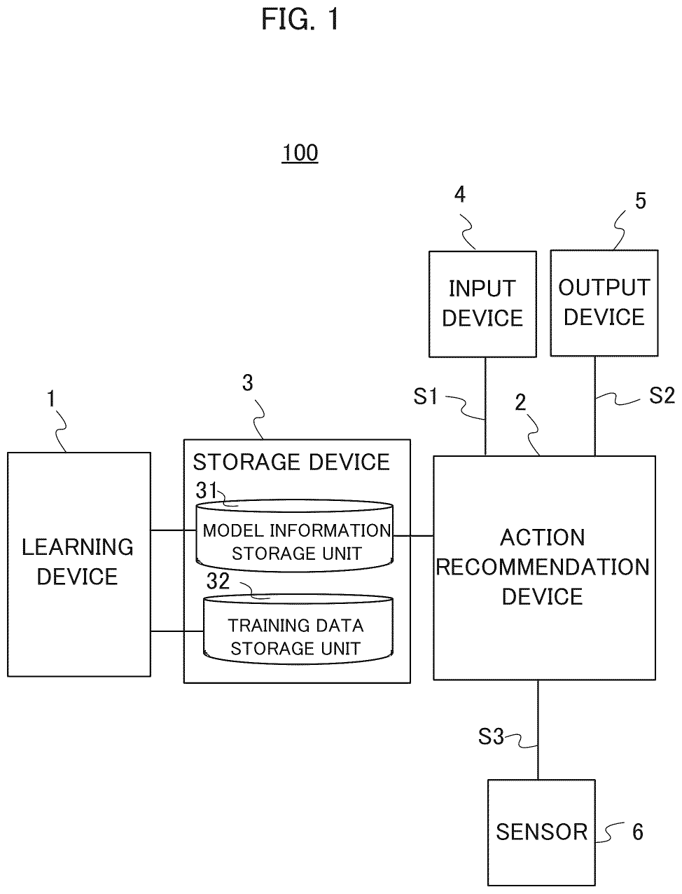

FIG. 1 shows a schematic configuration of an action recommendation system 100 according to the first example embodiment. The action recommendation system 100 is a system for health management of a target person, and trains a recommendation model configured to recommend the next action from the history of the target person's actions and health states and recommends the action using the trained recommendation model. As will be described later, the recommendation model is a model which learned the relation between history information indicating the history which associates each action with the health state of the person who took the each action and the recommended action to be taken by the person who took the each action.

Thereafter, the “target person” is a person subject to recommendation of an action by the action recommendation system 100 and it may be an individual user or a person whose activities are managed by an organization. Examples of the above-mentioned “health management” include not only a general health management such as a diet support for the purpose of improving the weight and body fat percentage and a health promotion for the purpose of improving test items such as blood glucose level, but also a condition maintenance of special business personnel such as athletes, management of rehabilitation of patients requiring rehabilitation. The term “action” includes any action that affects the health of the target person, and is not limited to active actions performed by her (his) self, but also includes passive actions such as undergoing massage or treatment.

The action recommendation system 100 mainly includes a learning device 1, an action recommendation device 2, a storage device 3, an input device 4, an output device 5, and a sensor 6. Here, data communication via a communication network or by wireless or wired direct communication is performed between the learning device 1 and the storage device 3, and between the action recommendation device 2 and the storage device 3. Similarly, data communication via a communication network or directly via wireless or wired communication is performed between the action recommendation device 2 and the input device 4, between the action recommendation device 2 and the output device 5, and between the action recommendation device 2 and the sensor 6.

The learning device 1 performs machine learning of the recommendation model as a learning device based on the training data stored in the training data storage unit 32 of the storage device 3, and stores the parameters of the recommendation model obtained through machine learning in the model information storage unit 31 of the storage device 3. Here, the recommendation model is a learning model which accepts information (also referred to as “action/state history information”) indicating the history of the action and health state of the target person as input data and which outputs the action (also referred to as “recommended action”) to be recommended to the target person as the inference result. As an exemplary machine learning method of such a recommendation model, in the present example embodiment, the above-described recommendation model is trained based on the SAIL (Skill Acquisition Learning) method. The details of the learning of the recommendation model will be described later.

The action recommendation device 2 configures the recommendation model based on the parameters stored in the model information storage unit 31 of the storage device 3, and determines the recommended action recommended to the target person based on the configured recommendation model and the action/state history information indicating the history of the preceding action and the health state of the target person. In this instance, the action recommendation device 2 acquires the action/state history information indicating the history of the preceding action and the health state of the target person, based on the input signal “S1” supplied from the input device 4, the sensor (detection) signal “S3” supplied from the sensor 6, or/and the information stored in the storage device 3. Then, the action recommendation device 2 outputs information on the determined recommended action by the output device 5. In this instance, the action recommendation device 2 generates an output signal “S2” related to the recommended action recommended to the target person and supplies the generated output signal S2 to the output device 5.

The input device 4 is an interface that accepts manual input (external input) of information regarding each target person. The user who operates the input device 4 to input information may be the target person itself, or may be a person who manages or supervises the activity of the target person. The input device 4 may be a variety of user input interfaces such as, for example, a touch panel, a button, a keyboard, a mouse, or a voice input device. The input device 4 supplies the generated input signal S1 to the action recommendation device 2. The output device 5 displays or outputs the predetermined data based on the output signal S2 supplied from the action recommendation device 2. Examples of the output device 5 include a display, a projector, a speaker and the like.

The sensor 6 measures a biometric signal or the like of the target person and supplies the measured biometric signal or the like to the action recommendation device 2 as a sensor signal S3. In this instance, the sensor signal S3 may be any biological signal (including vital information) regarding the target person such as heart rate, EEG, pulse wave, amount of sweat (skin electrical activity), amount of hormone secretion, cerebral blood flow, blood pressure, body temperature, myoelectricity, respiration rate, and acceleration. The sensor 6 may also be a device that analyzes blood collected from the target person and provides a sensor signal S3 indicative of the analysis result. The sensor 6 may be a wearable terminal worn by the target person, a camera for photographing the target person, a microphone for generating a voice signal of the target person, or a terminal such as a personal computer or a smartphone operated by the target person. Examples of the above-described wearable terminal includes a GNSS (Global Navigation Satellite System) receiver, an accelerometer, and a sensor for detecting a biological signal, and the wearable terminal outputs output signals of the respective sensors as a sensor signal S3. The sensor 6 may supply information corresponding to the amount of manipulation of a personal computer or a smartphone to the action recommendation device 2 as a sensor signal S3. The sensor 6 may also output a sensor signal S3 indicating biomedical data (including sleep time) detected from the target person during sleep of the target person.

The storage device 3 is a memory which stores various information necessary for processing performed by the learning device 1 and the action recommendation device 2. The storage device 3 may be an external storage device, such as a hard disk, connected to the learning device 1 and the action recommendation device 2 or embedded in either, or may be a storage medium, such as a portable flash memory. The storage device 3 may be a server device that performs data communication with the learning device 1 and the action recommendation device 2. Further, the storage device 3 may be configured by a plurality of devices.

The storage device 3 functionally includes a model information storage unit 31 and a training data storage unit 32.

The model information storage unit 31 stores the parameters of the recommendation model trained by the learning device 1. The recommendation model is trained so as to output, upon accepting the action/state history information indicating the history of the previous action and health state of a target person as input data, the recommended action to be recommended to the target person as an inference result. The parameters of the recommendation model are generated through machine learning, using action/state history information as input data while using labels (also referred to as “success/failure information”), each of which indicates either a success example (i.e., positive example) of the history of the action and the health state or a failure example (i.e., negative example) of the history of the action and the health state. Whether it is a success example or a failure example is determined based on whether or not the health state of the target person is improved (and the degree of improvement). Specifically, it is determined, prior to learning, based on an indicator (KPI: Key Performance Indicator) that is important for health care of the target person. The KPI is an example of the “benchmark indicator”.

In the case where a model based on a neural network such as a convolutional neural network is used as the recommendation model, various parameters regarding a layer structure adopted in the model, a neuron structure of each layer, the number of filters and the size of filters in each layer, and the weight for each element of each filter is stored in the model information storage unit 31. The parameters stored in the model information storage unit 31 is generated and updated by the learning device 1.

The training data storage unit 32 stores training data (learning data) which is used for learning (for training) by the learning device 1. The training data includes multiple sets of action/state history information indicating the action and health state of a subject (also referred to as “training subject”) for training data generation and a positive/negative label for the action/state history information.

The configuration of the action recommendation system 100 shown in FIG. 1 is an example, and various changes may be made to the configuration. For example, at least two of the learning device 1, the action recommendation device 2, and the storage device 3 may be realized by a single device. In another example, the learning device 1 and the action recommendation device 2 may be configured by a plurality of devices, respectively. In this case, the plurality of devices constituting the learning device 1 and the plurality of devices constituting the action recommendation device 2 exchange information necessary for executing preassigned process among them by wired or wireless direct communication or by communication through a network. In this case, the learning device 1 functions as a learning system, and the action recommendation device 2 functions as an action recommendation system. In yet another example, the input device 4 and the output device 5 may be integrally configured. In this case, the input device 4 and the output device 5 may be configured as a tablet-type terminal that is integral to or separate from the action recommendation device 2. Further, the input device 4 and the sensor 6 may be configured integrally.

### (2) Hardware Configuration

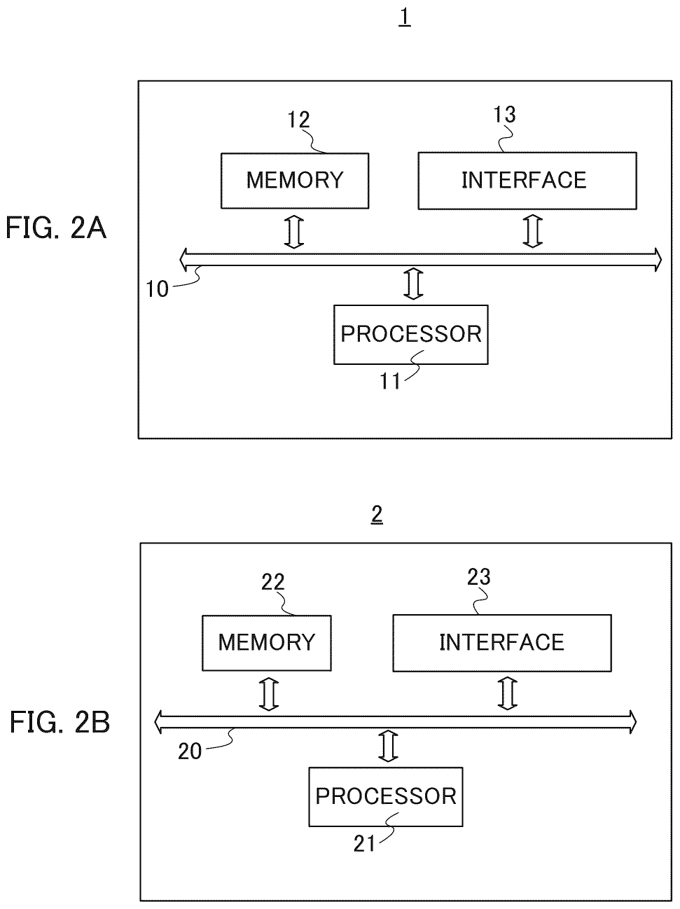

FIG. 2A shows a hardware configuration of the learning device 1. The learning device 1 includes a processor 11, a memory 12, and an interface 13 as hardware. The processor 11, memory 12 and interface 13 are connected to one another via a data bus 10.

The processor 11 functions as a controller (computing unit) for controlling the entire learning device 1 by executing a program stored in the memory 12. Examples of the processor 11 include a CPU (Central Processing Unit), a GPU (Graphics Processing Unit), and a TPU (Tensor Processing Unit). The processor 11 may be configured by a plurality of processors. The processor 11 is an example of a computer.

The memory 12 includes a variety of volatile and non-volatile memories, such as a RAM (Random Access Memory), a ROM (Read Only Memory), and a flash memory. Further, a program for executing a process executed by the learning device 1 is stored in the memory 12. A part of the information stored in the memory 12 may be stored in one or more external storage devices that can communicate with the learning device 1, and may be stored in a storage medium detachable from the learning device 1.

The interface 13 is an interface for electrically connecting the learning device 1 to other devices. Examples of the interfaces include a wireless interface, such as a network adapter, for transmitting and receiving data to and from other devices wirelessly, and a hardware interface, such as a cable, for connecting to other devices.

The hardware configuration of the learning device 1 is not limited to the configuration shown in FIG. 2A. For example, the learning device 1 may further include a display unit such as a display, an input unit such as a keyboard and a mouse, a sound output unit such as a speaker, or the like.

FIG. 2B shows an example of a hardware configuration of the action recommendation device 2. The action recommendation device 2 includes a processor 21, a memory 22, and an interface 23 as hardware. The processor 21, the memory 22, and the interface 23 are connected to one another via a data bus 20.

The processor 21 functions as a controller (computer) for controlling the entire action recommendation device 2 by executing a program stored in the memory 22. Examples of the processor 21 include a CPU, a GPU, a TPU, and a quantum processor. The processor 21 may be configured by a plurality of processors. The processor 21 is an example of a computer.

The memory 22 is configured by various volatile and non-volatile memories such as a RAM, a ROM, and a flash memory. Further, a program for executing the process performed by the action recommendation device 2 is stored in the memory 22. A portion of the information stored in the memory 22 may be stored in an external storage device (e.g., the memory device 3) that can communicate with the action recommendation device 2, or may be stored in a storage medium detachable from the action recommendation device 2. The memory 22 may also store information stored in the storage device 3 instead.

The interface 23 is one or more interfaces for electrically connecting the action recommendation device 2 to other devices. Examples of the interfaces include a wireless interface, such as a network adapter, for transmitting and receiving data to and from other devices wirelessly, and a hardware interface, such as a cable, for connecting to other devices.

The hardware configuration of the action recommendation device 2 is not limited to the configuration shown in FIG. 2B. For example, instead of connecting through the interface 23 to the input device 4, the output device 5, and the sensor 6, the action recommendation device 2 may incorporate at least one of them.

### (3) Overview of Recommendation Model

### (3-1) Basic Explanation of SAiL Method

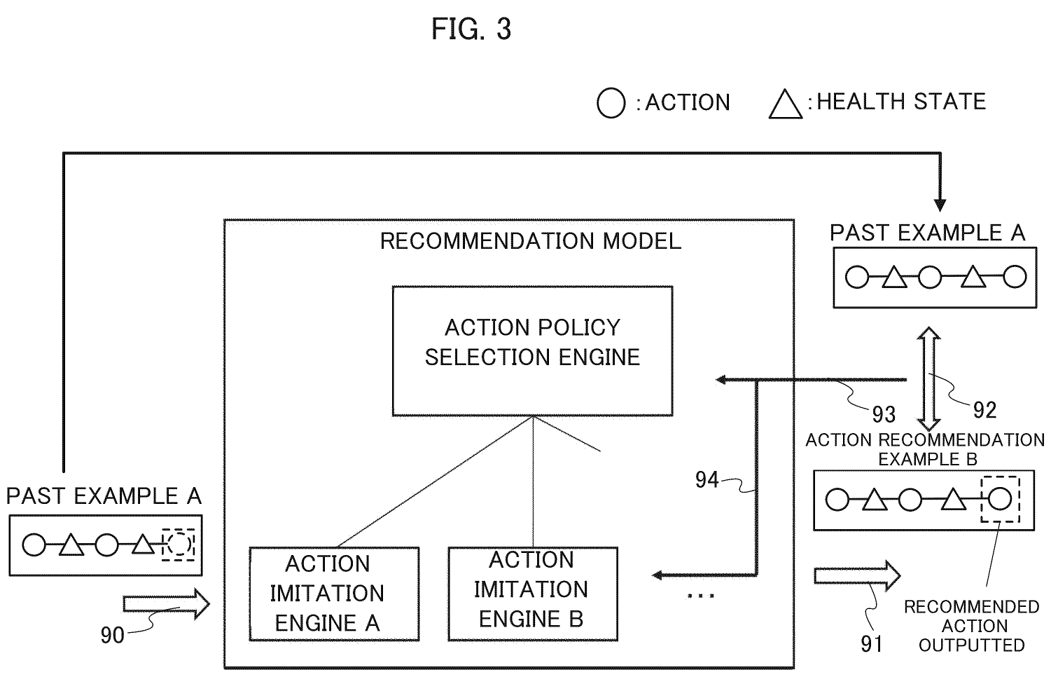

Next, a basic explanation will be given of the SAIL method used for training the recommendation model in the present example embodiment. FIG. 3 is a diagram schematically illustrating the behavior of generating the recommendation model according to the SAIL method. As shown in FIG. 3, the recommendation model includes an action policy selection engine and multiple action imitation engines (action imitation engine A, action imitation engine B, . . . ). Here, the circle (o) represents the action taken by a training subject, and the triangle (A) represents the health state of the training subject.

Here, each action imitation engine is a model configured to output, upon receiving a past example as an input, an action recommendation example which includes the inputted past example and a recommended action. In FIG. 3, the past example A indicative of a history with five elements in which an action and the health state as a result of the action are alternately indicated is used. The four elements except for the element of the last action of the past example A are inputted to each action imitation engine. Then, each action imitation engine infers the recommended action based on the inputted four elements of the past example A, and outputs the action recommendation example B which includes the inputted four elements of the past example A and the recommended action. Here, the action recommendation example B indicates a history with a total of five elements of actions and health states, including the inferred recommended action. The arrow 90 shown below the past example A on the left shows the input of the four elements excluding the last action of the past example A to each action imitation engine, and the arrow 91 under the action recommendation example B shows the output of the action recommendation example B from each action imitation engine.

When each action imitation engine infers an action recommendation example based on the inputted past example, the action policy selection engine compares the input (i.e., five elements of the past example A) with the inference result and selects the optimal action imitation engine based on the inference accuracy. The arrows 92 between the past example A and the action recommendation example B in FIG. 3 show a comparison between the inputted past example A with five elements, which is input, and the action recommendation example B, which is the inference result. The arrows 93 from arrow 92 to the action policy selection engine and the arrows 94 from arrows 92 to each action imitation engine indicate that the comparison result is inputted to the action policy selection engine and each action imitation engine.

Then, the learning device 1 generates the recommendation model by simultaneously training the action policy selection engine and the action imitation engine on the basis of the comparison result between the input and the inference result.

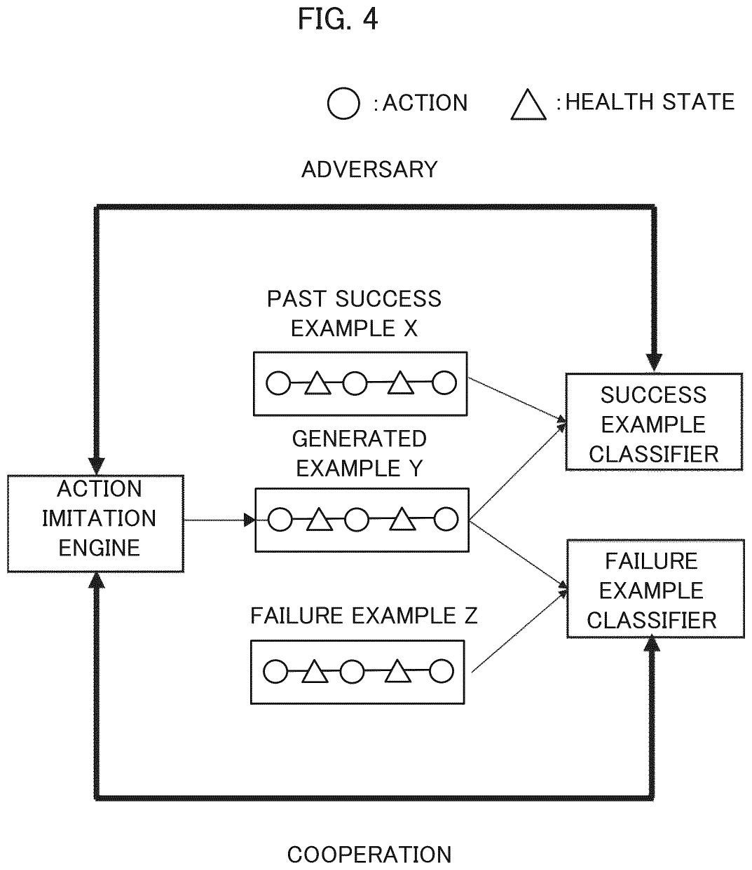

FIG. 4 is a diagram schematically illustrating a behavior of optimizing the action imitation engine. The learning device 1 generates the action imitation engine by ACIL (Adversarial Cooperative Imitation Learning) method. The learning device 1 causes the success example classifier that is a part of the action policy selection engine to compare an example generated by the action imitation engine with a past success example. Besides, the learning device 1 causes the failure example classifier that is a part of the action policy selection engine to compare an example generated by the action imitation engine with a past failure example. The past success example X in FIG. 4 is input data used as a positive example. In addition, the past failure example Z is input data used as a negative example. Whether the past example falls under a positive example or a negative example is identified by referring to the corresponding success/failure information. The generated example Y is data generated by the action imitation engine based on the input data.

A success example classifier distinguishes (or classifies) past success examples from examples generated by the action imitation engine. Therefore, the action imitation engine, which tries to make it approximate past success examples, and the success example classifier, which tries to distinguish it from past success examples, proceed with the learning (selection of the optimum action imitation engine) while antagonizing each other. The term “proceed with the learning while antagonizing each other” refers to the learning process to decrease the difference between the success example, which is input data, and the generated example, which is the inference result, wherein the action imitation engine tries to generate an example with little difference from success examples while the success example classifier tries to assess the little difference.

On the other hand, the failure example classifier distinguishes (or classifies) the past failure examples from examples generated by the action imitation engine. Therefore, the action imitation engine, which tries to distance it from past failure examples, and the failure example classifier, which tries to distinguish it from past failure examples, proceed with the learning while cooperating with each other. The term “proceed with the learning while cooperating with each other” refers to the learning process to increase the difference between the failure example, which is input data, and the generated example, which is the inference result, wherein the action imitation engine tries to generate an example with a large difference from failure examples while the failure example classifier tries to select an example with a larger difference. As described above, the learning device 1 performs machine learning using both the adversary and the cooperation, and thus it becomes possible to obtain a recommendation model capable of making highly accurate inference without leading to a fatal failure.

Detailed information regarding ACIL and SAIL methods can be found in Lu Wand et al., “Adversarial Cooperative Imitation Learning for Dynamic Treatment Regimes”, Proceedings of The Web Conference 2020 (WWW '20), [Search on Aug. 5, 2021] Internet <URL: https://dl.acm.org/doi/10.1145/3366423.3380248>.

In the example shown in FIGS. 3 and 4, the learning device 1 generates the recommendation model through machine learning using the training data of positive examples and negative examples, but the learning device 1 may generate the recommendation model through machine learning using only the training data of positive examples.

### (3-2) Action/State History Information

Next, the action/state history information used as input data to the recommendation device model in training the recommendation model or in determining the recommended action using the recommendation device model will be specifically described.

The action/state history information indicates a history alternately indicating the action and health state of the target person or the training subject. Hereafter, in the action/state history information, information indicating an action (i.e., an action corresponding to a circle (o) in FIGS. 3 and 4) at a certain time is referred to as “action element information”. The above-mentioned “time” may be a duration (e.g., time with a temporal width in units of hours to days). Further, in the action/state history information, the information indicating a health state at a certain time (i.e., a health state corresponding to a triangle (A) in FIGS. 3 and 4) is referred to as “state element information”.

The action indicated by the action element information is either an action whose timing (time slot, time, time of the state transition of the target person to a predetermined state) and content are detectable by the sensor 6 or the like, or, an action recordable as a record by manual input using the input device 4. Examples of such actions include actions related to exercise (e.g., walking, jogging, weight training, stretching, various types of sports, content of exercise, frequency of exercise, amount of exercise), actions related to eating (such as meal intake, dietary intake restrictions, and eating time), actions related to sleep (such as sleeping time and time slot), treatment (including body treatment and dosimetry) and massages. The action element information may include information indicating the type of action, or may indicates a combination of the type of action and the degree of action (amount of action).

Here, a description will be given of a case where the action element information indicates a combination of “type of action” and “degree of action”. For example, when the “type of action” is walking, the “degree of action” is the number of steps and/or the walking distance. In addition, when the “type of action” is a type of action relating to movement (e.g., jogging, weight training, and any other sports), the “degree of action” is distance, load, action time, and/or the like. Also, when the “type of action” is a limitation of calorie intake, the “degree of action” is the calorie intake or the amount of reduction on a daily basis. If the “type of action” is a type of eating action (sugar intake/intake limitation, protein intake/intake limitation, or any other nutrient intake/intake limitation, then the “degree of action” is the amount of nutrient intake or its reduction amount on a daily basis and/or an indicator (e.g., GI (Glycemic Index)) of eating intake. The action element information may not include information regarding the degree of action. In this case, the action element information indicates the type of action (e.g., workout and sugar intake restriction) that affects the health state.

The information (also referred to as “recommended action information”) regarding the recommended action outputted by the recommendation model indicates either the type of action or a combination of the type of action and the degree of action, as with the action element information. The recommended action information outputted by the recommendation model has the same data format as the action element information included in the action/state history information used for learning.

The state element information is information indicates a health state detectable by the sensor 6 or the like or a health state that can be acquired as a record by manual input to the input device 4 or the like and includes one or more health indicator values. Examples of the indicator values for health include a blood glucose value, a neutral fat value, a cholesterol value, any other value which can be acquired by blood exam, body weight, BMI, body fat rate, blood pressure, and heart rate. The state element data includes at least one or more indicator values of the health state used for calculation of the KPI, which is a key indicator in determining whether it is a success example or a failure example. Hereafter, the indicator values of the health state used for calculation of the above-mentioned KPI are also referred to as “KPI related indicator values”. The KPI related indicator value may be KPI itself.

The state element information may include not only indicator values required to calculate the KPI but also any indicator value(s) not directly used for calculation of the KPI. Hereafter, the indicator values relating to the health state that is not directly used for calculation of the KPI is also referred to as the “KPI peripheral indicator values”. For example, if the KPI is BMI, the KPI related indicator values are weight and height and the KPI peripheral indicator values are blood pressure, heart rate, blood glucose value, and the like. Thus, in some embodiments, the state element information includes not only the KPI related indicator values directly related to the KPI but also the KPI peripheral indicator values. Thereby, it is possible to train the recommendation model so as to output the optimal recommended action and determine the recommended action in comprehensive consideration of the health state of the target person.

### (3-3) Success/Failure Information

Next, the success/failure information used as labels in training the recommendation model will be described. The success/failure information is information indicating whether the action/state history information used as a counterpart in training the recommendation model is a success case (i.e., a positive example) or a failure case (i.e., a negative example), and is stored in the training data storage unit 32.

For example, if the KPI calculated by use of the state element information representing the latest health state, which is selected from the state element information included in action/state history information, belongs to a predetermined preferred value range, the success/failure information which indicates that the corresponding action/state history information is a positive example. In contrast, if the KPI calculated by use of the state element information representing the latest health state, which is selected from the state element information included in action/state history information, does not belong to the predetermined preferred value range, the success/failure information which indicates that the corresponding action/state history information is a negative example. On the assumption that the KPI is a body weight, the success/failure information is generated to indicate that it is a positive example if the body weight decreases after a series of the actions or after a certain period thereof, while it is generated to indicate that it is a negative example if the body weight does not decrease after a series of actions or after a certain period thereof.

The determination of the positive example or the negative example is not limited to being made only based on the latest (final) health state, but may be made in consideration of the health states in the process leading to the latest health state. In this case, conditions may be respectively provided for the latest health state and the intermediate health states, and success/failure information indicating that it is a positive example may be given to the action/state history information in which those conditions are satisfied. In some embodiments, success/failure information may be generated based on any rules or genuine techniques other than the generation example described above.

### (4) Process by Learning Device

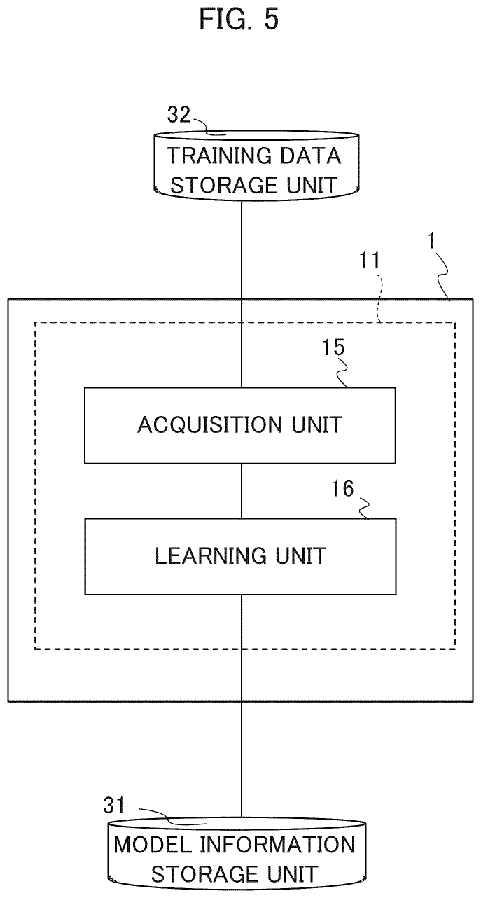

FIG. 5 is an example of functional blocks of the learning device 1. The processor 11 of the learning device 1 functionally includes an acquisition unit 15 and a learning unit 16.

The acquisition unit 15 acquires a set of the action/state history information and the success/failure information that have not yet been used for learning of the recommendation model from the training data storage unit 32 through the interface 13 and supplies the acquired set of the action/state history information and the success/failure information to the learning unit 16. The acquisition unit 15 acquires the set of the action/state history information and the success/failure information and supplies it to the learning unit 16 until the learning unit 16 completes the learning of the recommendation model or until all sets of the action/state history information and the success/failure information stored in the training data storage unit 32 are acquired.

The learning unit 16 trains the recommendation model based on the set of the action/state history information and the success/failure information supplied from the acquisition unit 15. Specifically, the learning unit 16 determines whether the action/state history information is a success example or a failure example based on the success/failure information, and further trains the action policy selection engine (including the success example classifier and the failure example classifier) and the action imitation engine so as to output the inference result that is close to the success example while being far away from the failure example, according to the SAIL method illustrated in FIGS. 3 and 4. Then, the learning unit 16 updates the parameters of the action policy selection engine and the action imitation engine by any parameter determination algorithm such as a gradient descent method and an error back propagation method, and stores the updated parameters in the model information storage unit 31. If a predetermined condition for ending learning is satisfied, the learning unit 16 completes the learning of the recommendation model. For example, if the learning using a predetermined sets of the action/state history information and the success/failure information is completed, if the learning unit 16 detects a user input or the like indicating that the learning should be terminated, and/or if the error in the training becomes equal to or less than a predetermined threshold value, the learning unit 16 determines that the termination condition of the learning is satisfied.

Each component of the acquisition unit 15 and the learning unit 16 can be realized, for example, by the processor 11 executing a program. In addition, the necessary program may be recorded in any non-volatile storage medium and installed as necessary to realize the respective components. In addition, at least a part of these components is not limited to being realized by a software program and may be realized by any combination of hardware, firmware, and software. At least some of these components may also be implemented using user-programmable integrated circuitry, such as FPGA (Field-Programmable Gate Array) and microcontrollers. In this case, the integrated circuit may be used to realize a program for configuring each of the above-described components. Further, at least a part of the components may be configured by a ASSP (Application Specific Standard Produce), ASIC (Application Specific Integrated Circuit) and/or a quantum processor (quantum computer control chip). In this way, each component may be implemented by a variety of hardware. The above is true for other example embodiments to be described later. Further, each of these components may be realized by the collaboration of a plurality of computers, for example, using cloud computing technology.

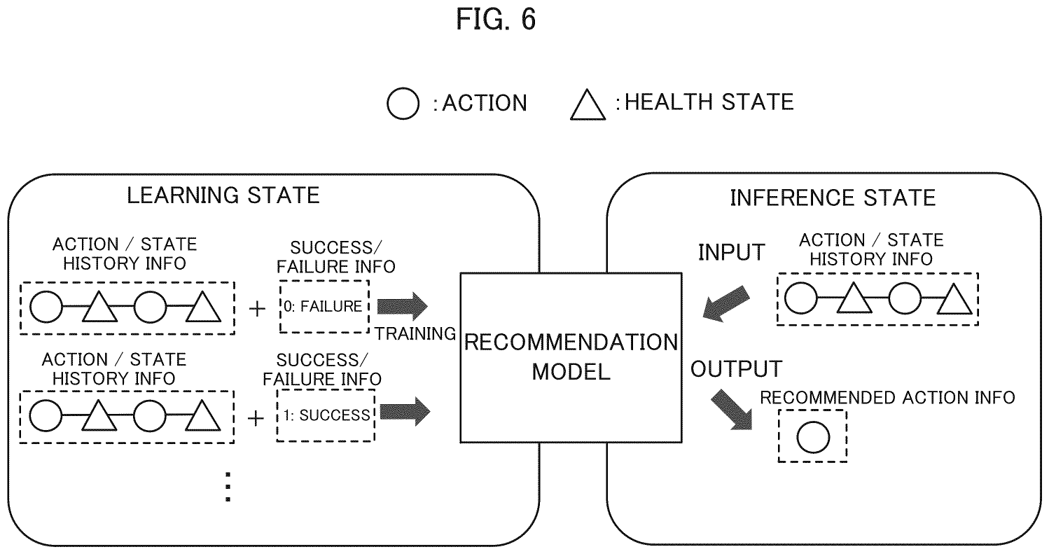

FIG. 6 is a diagram schematically illustrating learning of the recommendation model and calculation of the recommended action using the recommendation model. As shown in the drawing, the learning unit 16 trains the recommendation model by inputting a set of the action/state history information and the success/failure information into the recommendation model in the learning stage. In this case, the learning unit 16 trains the recommendation model using the action/state history information which falls under a failure example (here, with the success/failure information indicative of “0”) and the action/state history information which falls under a success example (here, with the success/failure information indicative of “1”), respectively. Here, the action/state history information is configured by a total of four elements of action and health state, but it is not limited thereto and may be configured by a variable number of elements. Examples of the action/state history information used for learning include action/state history information with a total of two elements (i.e., a set of action and health state) and action/state history information with a total of six or more elements.

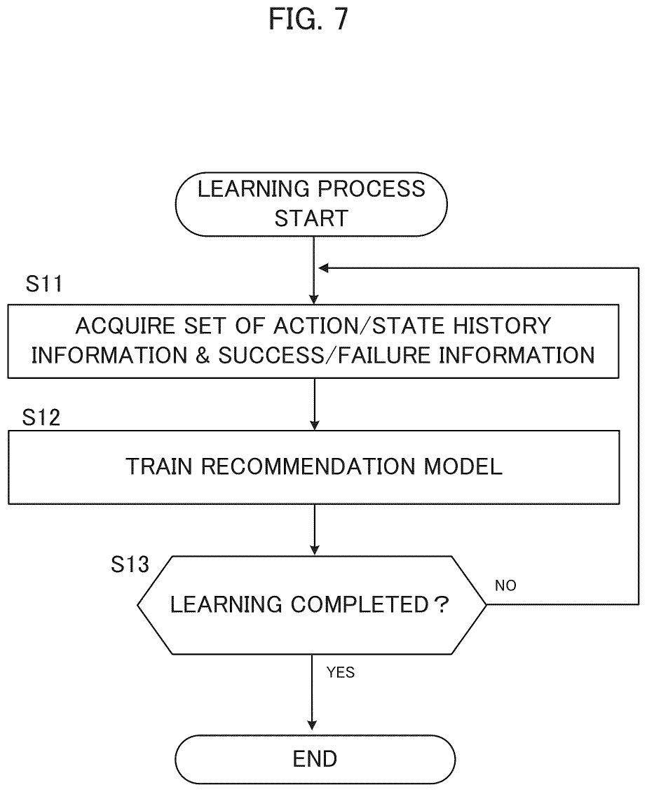

FIG. 7 is an example of a flowchart illustrating the learning process of the recommendation model that is executed by the learning device 1.

First, the learning device 1 acquires a set of the action/state history information and the success/failure information from the training data storage unit 32 (step S11). Then, the learning device 1 trains the recommendation model based on the set of the action/state history information and the success/failure information acquired at step S11 (step S12). In this case, the learning device 1 updates the parameters of the recommendation model based on the set of the action/state history information and the success/failure information acquired at step S11, and stores the updated parameters in the model information storage unit 31.

Then, the learning device 1 determines whether or not the learning is completed (step S13). Upon determining that the learning is completed (step S13; Yes), the learning device 1 terminates the process of the flowchart. On the other hand, upon determining that the learning has not been completed (step S13; No), the learning device 1 gets back to the process at step S11.

### (5) Process by Action Recommendation Device

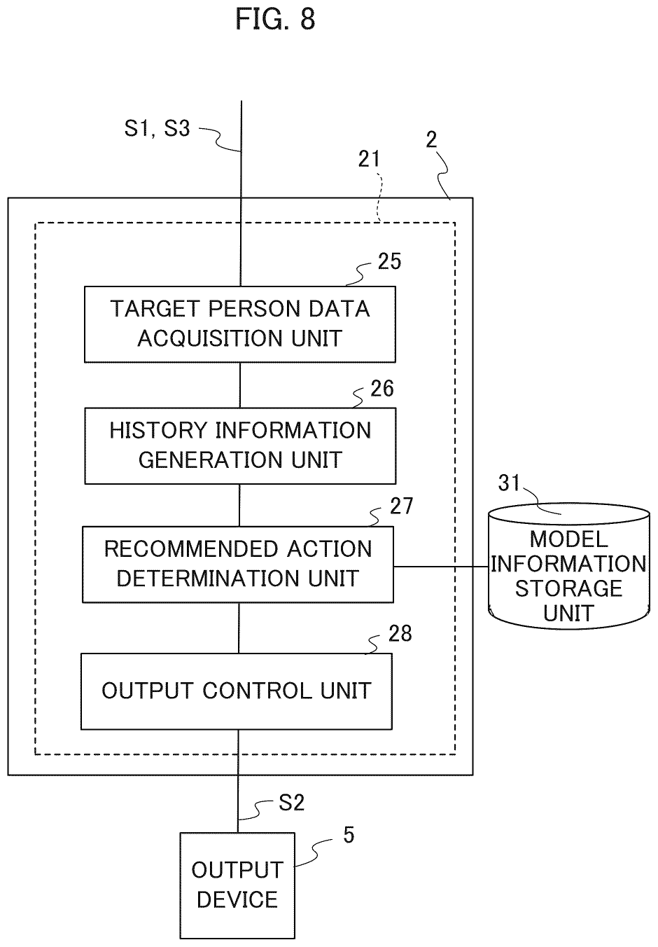

FIG. 8 is an example of the functional blocks of the action recommendation device 2. The processor 21 of the action recommendation device 2 functionally includes a target person data acquisition unit 25, a history information generation unit 26, a recommended action determination unit 27, and an output control unit 28. In FIG. 8, blocks to exchange data with each other are connected by a solid line, but the combination of blocks to exchanging data with each other is not limited thereto. The same applies to the drawings of other functional blocks described below.

The target person data acquisition unit 25 acquires data (also referred to as “target person data”) relating to the target person necessary for generating the action/state history information (i.e., the action element information and the state element information) regarding the target person through the interface 23. In this instance, the target person data acquisition unit 25 acquires the input signal S1 generated by the input device 4 and/or the sensor signal S3 generated by the sensor 6. If any attribute information (age, height, weight, and the like) relating to the target person necessary for generating the action/state history information is stored in the storage device 3, the target person data acquisition unit 25 or the history information generation unit 26 to be described later may acquire the attribute information regarding the target person necessary for generating the action/state history information from the storage device 3. The target person data acquired by the target person data acquisition unit 25 may be stored in the storage device 3, the memory 22, or the like in association with, for example, the acquisition date and time acquired by the target person data acquisition unit 25 or the date and time specified by the user.

The history information generation unit 26 extracts the action and health state of the target person in time series from the time series target person data acquired by the target person data acquisition unit 25, and generates the action element information and the state element information in time series based on the extraction result. Then, the history information generation unit 26 supplies the action/state history information as time series data of the generated action element information and the state element information to the recommended action determination unit 27.

In this case, if the target person data is the sensor signal S3, the history information generation unit 26 performs a predetermined feature extracting process on the sensor signal S3 to calculate indicators (KPI related indicator and KPI peripheral indicator) representing the health state of the target person. For example, if the sensor signal S3 is biomedical data such as heart rate or amount of perspiration, the history information generation unit 26 performs a predetermined feature extracting process on the biomedical data and a stress estimation process based on the extracted features to calculate a stress value that is a KPI related indicator value or a KPI peripheral indicator value. Various techniques have been proposed for estimating the degree of stress from biological data. In another example, if the sensor signal S3 is output data such as an acceleration sensor of a smartphone or a wearable sensor, the history information generation unit 26 counts the number of steps in the predetermined period according to the sensor signal S3 acquired in the predetermined period and generates the action element information representing the number of steps in the predetermined period based on the counting result.

As described above, the history information generation unit 26 performs a feature extraction process (including a feature extraction technique using a learning model based on a neural network or the like) to convert the target person data into the action element information and the state element information in a data format which conforms to the input format of the recommendation model. Then, the history information generation unit 26 supplies the action/state history information including the action element information and the state element information in time series to the recommended action determination unit 27. In this case, for example, the action/state history information is represented by a tensor in a predetermined format.

Here, a supplementary description will be given of a method of generating action element information and state element information in time series. For example, the history information generation unit 26 sets each observation period of the action targeted in each action element information included in the action/state history information, and generates the action element information representing the target action for each observation period, based on the sensor signal S3 or the like obtained for each set observation period. Similarly, the history information generation unit 26 sets each observation timing of the health state targeted in the state element information included in the action/state history information, and generates the state element information representing the health state of the target person based on the sensor signal S3 or the like obtained at each observation timing. In this case, the observation timing of the health state is set, for example, immediately after the observation period of each action or an interval between observation periods of each action. Then, the history information generation unit 26 generates the action/state history information including a predetermined number of latest action element information and state element information generated in time series, and supplies the generated action/state history information to the recommended action determination unit 27.

The recommended action determination unit 27 acquires the parameters of the recommendation model from the model information storage unit 31, and inputs the action/state history information supplied from the history information generation unit 26 to the recommendation model configured based on the parameters, and acquires the recommended action information indicating the recommended action outputted by the recommendation model in response to the input of the action/state history information.

In some embodiments, the recommended action determination unit 27 acquires, in conjunction with the recommended action information, basis information regarding a basis (proof or ground) of the determination of the recommended action. For example, the basis information includes: a past recommended action to a person having similar action/state history information to the target person or to a person having a similar attribute to the target person; and the state of the person that resulted from the past recommended action. Specifically, if the recommended action determination unit 27 generates the recommended action information indicative of “walking for an hour per day” for the target person, such information as “because the person AA similar to the target person in health state has improved the health state by walking for an hour per day” will be generated as the basis information. This allows the target person to take the recommended action with a sense of satisfaction, thus increasing the possibility of taking the recommended action. The basis information may be generated by any other comportment using the recommendation model instead of the recommended action determination unit 27.

Here, the process executed by the recommended action determination unit 27 will be described supplementally with reference to FIG. 6 again. In the example shown in FIG. 6, the recommended action determination unit 27 inputs the action/state history information having four elements (the action, the health state, the action, and the health state) into the recommendation model, and acquires the recommended action information indicating the recommended action outputted from the recommendation model. The recommended action determination unit 27 supplies the acquired recommended action information to the output control unit 28. It should be noted that the action/state history information to be inputted to the recommendation model does not need to indicate the history in which the action is the first element and the health state is the last element as shown in FIG. 6, and may indicate the history in which the health state is the first element or the history in which the action is the last element.

A description will now be given of the output control unit 28 with reference to FIG. 8. The output control unit 28 outputs information regarding the determined recommended action by controlling the output device 5 based on the recommended action information supplied from the recommended action determination unit 27. In this instance, the recommended action determination unit 27 generates image information, text information, and/or voice information (collectively referred to as “recommended action promotion information”) for prompting the target person to take the recommended action as an output signal S2, and supplies the output signal S2 to the output device 5 by the output control unit 28, thereby causing the output device 5 to output the image information, text information, and/or voice information for prompting the recommended action. In this case, for example, the output control unit 28 notifies the user of the text information to the effect that “Please reduce the calorie intake (or fat, sugar) by Z” (Z is a positive number) and “Please increase the number of steps by V” (V is a positive number) based on the recommended action promotion information.

Thus, the output control unit 28 can suitably recommend the action that the target person should take next. Instead of generating the recommended action promotion information as the output signal S2, the output control unit 28 may store the recommended action information or the recommended action promotion information in the storage device 3 or the memory 22 or may transmit the recommended action promotion information to an external device that performs data communication with the action recommendation device 2.

Each component of the target person data acquisition unit 25, the history information generation unit 26, the recommended action determination unit 27, and the output control unit 28 can be realized, for example, by the processor 11 executing a program. In addition, the necessary program may be recorded in any non-volatile storage medium and installed as necessary to realize the respective components. In addition, at least a part of these components is not limited to being realized by a software program and may be realized by any combination of hardware, firmware, and software. At least some of these components may also be implemented using user-programmable integrated circuitry, such as FPGA and microcontrollers. In this case, the integrated circuit may be used to realize a program for configuring each of the above-described components. Further, at least a part of the components may be configured by an ASSP, ASIC and/or a quantum processor (quantum computer control chip). In this way, each component may be implemented by a variety of hardware. The above is true for other example embodiments to be described later. Further, each of these components may be realized by the collaboration of a plurality of computers, for example, using cloud computing technology.

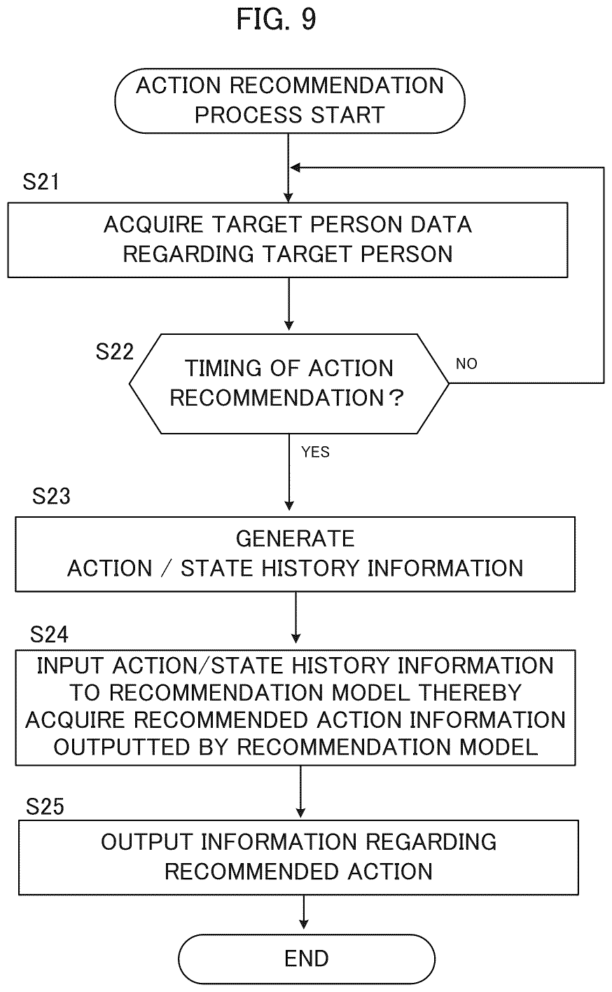

FIG. 9 is an example of a flowchart of an action recommendation process performed by the action recommendation device 2.

First, the action recommendation device 2 acquires the target person data regarding the target person from at least one of the input device 4, the sensor 6, and/or the storage device 3 (step S21). Then, the action recommendation device 2 determines whether or not a timing of making the action recommendation comes up (step S22). For example, upon receiving the input signal S1 for requesting the action recommendation from the input device 4 or upon determining that now is a predetermined time or time slot to make the action recommendation or upon determining that any other predetermined action recommendation execution condition is met, the action recommendation device 2 determines that timing of making the action recommendation has come up. In this case, the action recommendation execution condition may be determined based on the health state of the target person. Upon detecting a predetermined health state (e.g., when the detected stress value of the target person becomes a predetermined value or more) or upon detecting a health state determined to be necessary to make an action recommendation, the action recommendation device 2 may determine that the action recommendation execution condition is satisfied, for example. The processor 21 of the action recommendation device 2 serves as a “determination means” at step S22.

Upon determining that a timing of making the action recommendation does not come up (step S22; No), the action recommendation device 2 performs a process of acquiring the target person data at step S21.

On the other hand, upon determining that a timing of the action recommendation has come up (step S22; Yes), the action recommendation device 2 generates the action/state history information based on the target person data acquired at step S21 (step S23). Then, the action recommendation device 2 inputs the action/state history information generated at step S23 to the recommendation model configured by referring to the model information storage unit 31 and acquires the recommended action information indicating the recommended action outputted by the recommendation model (step S24). The action recommendation device 2 outputs the recommended action recommended to the target person by the output device 5 based on the recommended action information acquired at step S24 (step S25).

### (6) Application Example

Here, an application example will be described. As an application, the target person is a medical checkup examinee, and the action recommendation device 2 recommends the action to be taken by the target person based on the data of the medical checkup regularly carried out on a yearly or monthly basis. In this case, the diagnosis data is a diagnosis result or a measurement result of each diagnosis item such as a height, a body weight, a blood test result, a urine test result, an X-ray test result, and an electrocardiogram, and the diagnosis data thereof is stored in the storage device 3.

Upon determining that action recommendation should be made at step S22 in FIG. 9, the learning device 1 acquires the diagnosis data of the medical checkup of the target person by referring to the storage device 3 and generates state element information indicating the health state at the medical checkup of the target person from the diagnosis data. In addition, the learning device 1 generates, based on the target person data observed prior to the medical checkup, the action element information indicating the action prior to the medical checkup in the same manner as in the above-described example embodiment, and generates, at step S23, the action/state history information including the generated action element information and the state element information based on the medical checkup result. After that, the learning device 1 acquires the recommended action information outputted by the recommendation model by inputting the action/state history information into the recommendation model at step S24, and performs the output related to the recommended action at step S25.

Thus, according to an application example, the learning device 1 can suitably recommend an action to be taken by a target person, who is a medical checkup examinee, to the target person based on the diagnosis data of the medical checkup. The learning device 1 may generate state element information representing the health state of the target person using both the diagnosis data of the medical checkup and the sensor signal S3 outputted by the wearable terminal or the sensor 6 provided in the portable terminal owned by the target person.

### (7) Modification

The learning device 1 trains the recommendation model for each class of the attribute of training subjects, and the action recommendation device 2 may select the recommendation model for determining the recommended action based on the attribute of the target person.

In this case, the training subjects are classified into a plurality of groups based on a predetermined attribute (e.g., age, gender, race, etc.), and the learning device 1 trains the recommendation model for each group based on the training data corresponding to the training subjects divided for each group, and stores parameters of the recommendation model for each group obtained by learning in the model information storage unit 31. Then, when determining the recommended action of the target person, the action recommendation device 2 recognizes the attribute of the target person based on the attribute information of the target person stored in the storage device 3 or the signal acquired from the input device 4 or the sensor 6, and recognizes the group in which the target person is classified based on the recognized attribute of the target person. Then, the storage device 3 extracts the parameters of the recommendation model corresponding to the group in which the target person is classified from the model information storage unit 31 to configure the recommendation model, and determines the recommended action of the target person using the recommendation model.

In this way, the action recommendation system 100 according to the modification can learn the action that the target person should take based on the training data acquired from the training subjects having the similar attribute to the target person and recommend the action more suitable for the target person to the target person.

### Second Example Embodiment

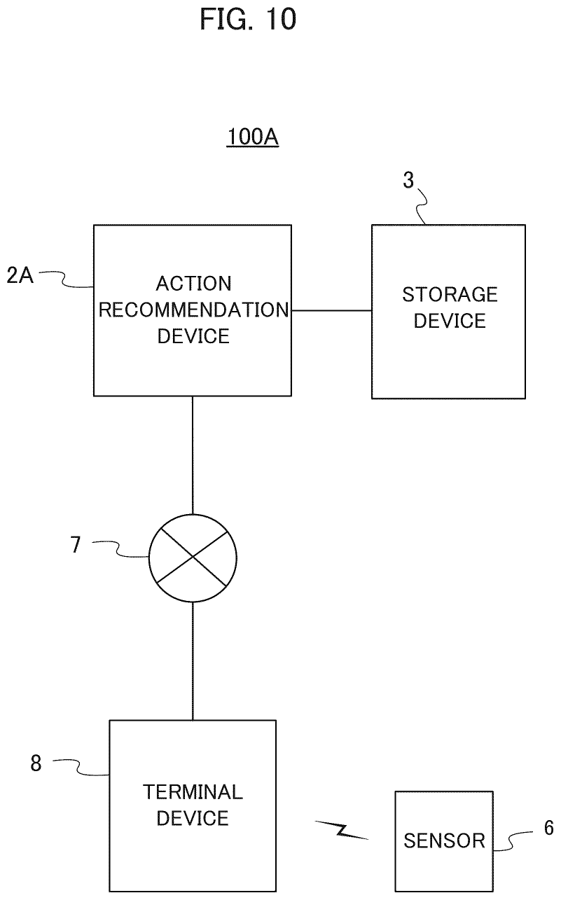

FIG. 10 shows a schematic configuration of an action recommendation 100A according to the second example embodiment. The action recommendation system 100A according to the second example embodiment is a system according to a server-client model, and an action recommendation device 2A functioning as a server device executes the processes executed by the learning device 1 and the action recommendation device 2 according to the first example embodiment. Hereinafter, the same components as in the first example embodiment are appropriately denoted by the same reference numerals, and a description thereof will be omitted.

As shown in FIG. 10, the action recommendation system 100A mainly includes an action recommendation device 2A that functions as a server, a storage device 3 that stores substantially the same data as the data stored in the storage device 3 in the first example embodiment, and a terminal device 8 that functions as a client. The action recommendation device 2A and the terminal device 8 performs data communication via the network 7 with each other.

The terminal device 8 is a terminal having an input function, a display function, and a communication function, and functions as the input device 4 and the output device 5 shown in FIG. 1. Examples of the terminal device 8 include a personal computer, a tablet-type terminal, and a PDA (Personal Digital Assistant). The terminal device 8 transmits a biometric signal outputted by the sensor 6 or an input signal based on a user input to the action recommendation device 2A.

The action recommendation device 2A is equipped with a hardware configuration shown in FIG. 2A or FIG. 2B and functional block configuration respectively shown in FIG. 5 and FIG. 8. After the learning process of the recommendation model shown by the flowchart in FIG. 7 or the like, the action recommendation device 2A receives the information on the target person that the action recommendation device 2 shown in FIG. 1 acquires from the input device and the sensor 6 as the target person data from the terminal device 8 through the network 7, and executes the action recommendation process shown by the flowchart in FIG. 9 or the like on the basis of the received target person data. In this instance, the action recommendation device 2A (specifically, the output control unit 28 in FIG. 8) transmits an output signal related to the recommended action determined by the action recommendation process to the terminal device 8 through the network 7 based on a request from the terminal device 8. In this case, the terminal device 8 functions as an output device 5 in the first example embodiment.

As described above, the action recommendation device 2A according to the second example embodiment can suitably present the user of the terminal device 8 with information on the recommended action determined based on the history of the action and the healthy state of the user of the terminal device 8. In the second example embodiment, a device different from the action recommendation device 2A may perform the learning process of the recommendation model.

### Third Example Embodiment

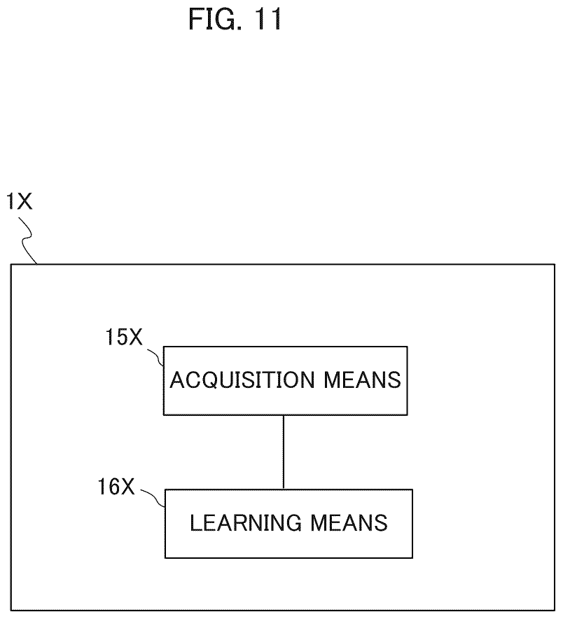

FIG. 11 is a block diagram of a learning device 1X according to the third example embodiment. The learning device 1X mainly includes an acquisition means 15X and a learning means 16X. The learning device 1X may be configured by a plurality of devices.

The acquisition means 15X is configured to acquire history information indicating a history of a health state of a target person and an action of the target person contributing to variation in the health state, and success/failure information indicating whether or not the action contributed to the variation in the health state of the target person. Examples of the acquisition means 15X include the acquisition unit 15 of the learning device 1 in the first example embodiment and the acquisition unit 15 of the action recommendation device 2A in the second example embodiment.

The learning means 16X is configured to train a model based on the history information and the success/failure information, wherein the model is configured to output information regarding a recommended action recommended to improve the health state of the target person upon receiving an input of the history information indicating the history of the action and the health state of the target person. Examples of the learning means 16X include the learning unit 16 of the learning device 1 in the first example embodiment and the learning unit 16 of the action recommendation device 2A in the second example embodiment.

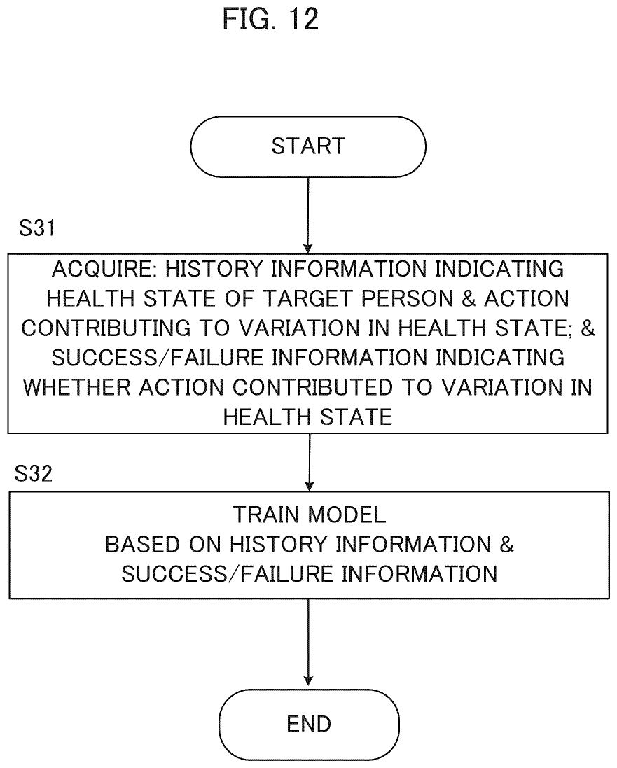

FIG. 12 is an example of the flowchart that is executed by the learning device 1X in the third example embodiment. The acquisition means 15X of the learning device 1X acquires history information indicating a history of a health state of a target person and an action of the target person contributing to variation in the health state, and success/failure information indicating whether or not the action contributed to the variation in the health state of the target person (step S31). Then, the learning means 16X of the learning device 1X trains a model based on the history information and the success/failure information, wherein the model is configured to output information regarding a recommended action recommended to improve the health state of the target person upon receiving an input of the history information indicating the history of the action and the health state of the target person (step S32).

According to the third example embodiment, it is possible to train a model capable of determining a recommended action to be recommended to a target person in consideration of a history of the action of the target person and the health state thereof.

### Fourth Example Embodiment

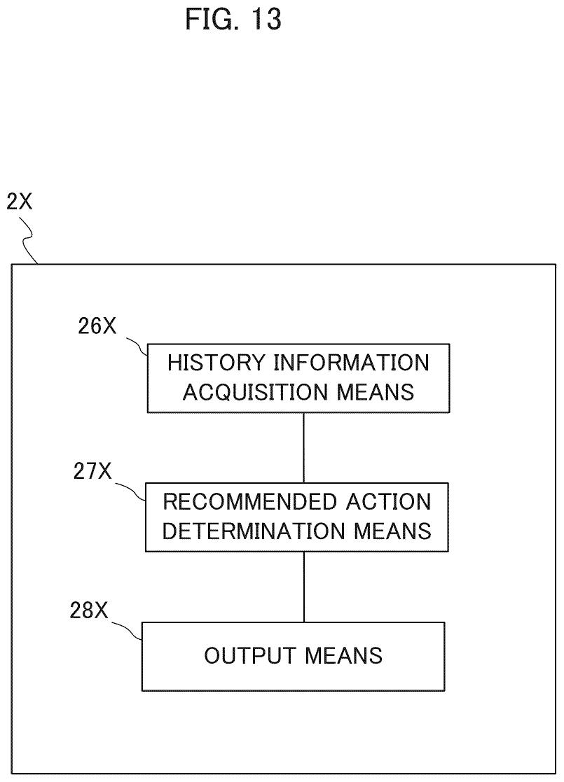

FIG. 13 is a block diagram of the action recommendation device 2X according to the fourth example embodiment. The action recommendation device 2X mainly includes a history acquisition means 26X, a recommended action determination means 27X, and an output means 28X. The action recommendation device 2Y may be configured by a plurality of devices.

The history information acquisition means 26X is configured to acquire history information indicating a history of a health state of a target person and an action of the target person contributing to variation in the health state. Examples of the history information acquisition means 26X include the history information generation unit 26 of the action recommendation device 2 according to the first example embodiment and the history information generation unit 26 of the action recommendation device 2A according to the second example embodiment.

The recommended action determination means 27X is configured to determine a recommended action to be recommended to the target person based on the history information and a recommendation model. The recommendation model is a model which learned a relation between each health state of subjects and each recommended action to be recommended to improve the each health state of the subjects, based on history information indicating histories of health states of the subjects and actions of the subjects which contribute to variation in the health states. Examples of the recommended action determination device 27X include the recommended action determination unit 27 of the action recommendation device 2 in the first example embodiment and the recommended action determination unit 27 of the action recommendation device 2A in the second example embodiment.

The output means 28X is configured to output information regarding the recommended action. In this case, the output means 28X may be configured to display and/or output information regarding the recommended action to the action recommendation device connected to the action recommendation device 2X by wire or wirelessly or to the output device built in the action recommendation device 2X, or may transmit the information regarding the recommended action to an external device connected to the action recommendation device 2X by wire or wirelessly, or may store the information regarding the recommended action in a storage device connected to the action recommendation device 2X by wire or wirelessly or built into the action recommendation device 2X. Examples of the output means 28X include the output control unit 28 of the action recommendation device 2 according to the first example embodiment and the output control unit 28 of the action recommendation device 2A according to the second example embodiment.

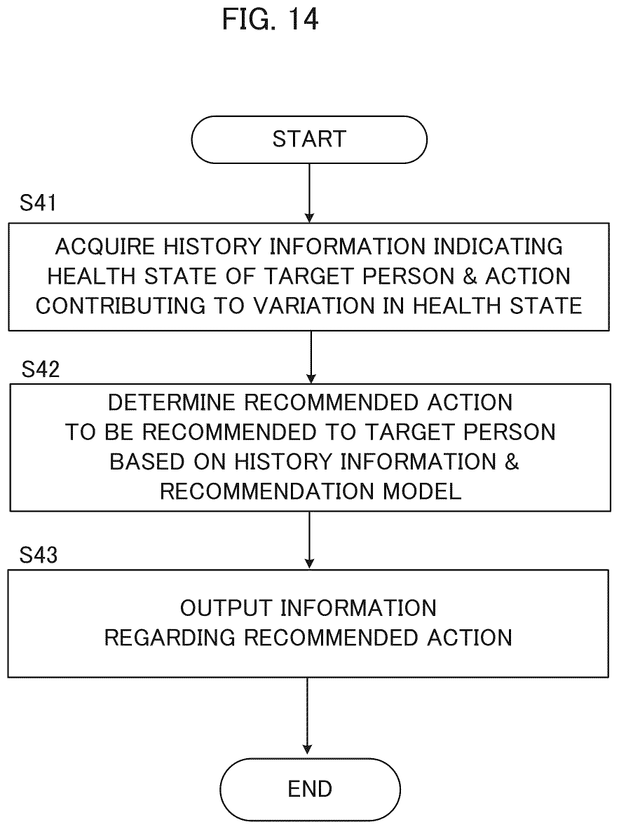

FIG. 14 is an example of the flowchart executed by the action recommendation device 2X in the fourth example embodiment. The history information acquisition means 26X of the the action recommendation device 2X acquires history information indicating a history of a health state of a target person and an action of the target person contributing to variation in the health state (step S41). The recommended action determination means 27X of the the action recommendation device 2X determines a recommended action to be recommended to the target person based on the history information and a recommendation model (step S42). The recommendation model is a model which learned a relation between each health state of subjects and each recommended action to be recommended to improve the each health state of the subjects, based on history information indicating histories of health states of the subjects and actions of the subjects which contribute to variation in the health states. Then, the output means 28X of the the action recommendation device 2X outputs information regarding the recommended action (step S43).

The action recommendation device 2X according to the fourth example embodiment allows for accurate determination and output of a recommended action to be recommended to the target person, taking into account the actional and health history of the target person.

In the example embodiments described above, the program is stored by any type of a non-transitory computer-readable medium (non-transitory computer readable medium) and can be supplied to a control unit or the like that is a computer. The non-transitory computer-readable medium include any type of a tangible storage medium. Examples of the non-transitory computer readable medium include a magnetic storage medium (e.g., a flexible disk, a magnetic tape, a hard disk drive), a magnetic-optical storage medium (e.g., a magnetic optical disk), CD-ROM (Read Only Memory), CD-R, CD-R/W, a solid-state memory (e.g., a mask ROM, a PROM (Programmable ROM), an EPROM (Erasable PROM), a flash ROM, a RAM (Random Access Memory)). The program may also be provided to the computer by any type of a transitory computer readable medium. Examples of the transitory computer readable medium include an electrical signal, an optical signal, and an electromagnetic wave. The transitory computer readable medium can provide the program to the computer through a wired channel such as wires and optical fibers or a wireless channel.

The whole or a part of the example embodiments described above can be described as, but not limited to, the following Supplementary Notes.

### [Supplementary Note 1]

A learning device comprising:
an acquisition means configured to acquire history information indicating a history of a health state of a target person and an action of the target person contributing to variation in the health state, and success/failure information indicating whether or not the action contributed to the variation in the health state of the target person; and
a learning means configured to train a model based on the history information and the success/failure information, wherein the model is configured to output information regarding a recommended action recommended to improve the health state of the target person upon receiving an input of the history information indicating the history of the action and the health state of the target person.

### [Supplementary Note 2]

The learning device according to Supplementary Note 1,
wherein the history information is information that indicates the action and the health state observed after the action alternately in time series.

### [Supplementary Note 3]

The learning device according to Supplementary Note 1 or 2,
wherein the history information includes, as a record of the action, information regarding a type of the action and a degree of the action.

### [Supplementary Note 4]

The learning device according to any one of Supplementary Notes 1 to 3,
wherein the history information includes, as a record of the health state, an indicator related to the health state used for calculation of a benchmark indicator used as a criterion for determining whether or not the history is the success example.

### [Supplementary Note 5]

An action recommendation device comprising:
a history information acquisition means configured to acquire history information indicating a history of a health state of a target person and an action of the target person contributing to variation in the health state;
a recommended action determination means configured to determine a recommended action to be recommended to the target person based on the history information and a recommendation model; and
an output means configured to output information regarding the recommended action,
wherein the recommendation model is a model which learned a relation between each health state of subjects and each recommended action to be recommended to improve the each health state of the subjects, based on history information indicating histories of health states of the subjects and actions of the subjects which contribute to variation in the health states.

### [Supplementary Note 6]

The action recommendation device according to Supplementary Note 5,
wherein the recommended action determination means is configured to generate recommended action promotion information for notifying the target person of the recommended action, and
wherein the output means is configured to further output the recommended action promotion information.

### [Supplementary Note 7]

The action recommendation device according to Supplementary Note 5 or 6, further comprising
a basis information generation means configured to generate basis information regarding a basis of determining the recommended action,
wherein the output means is configured to output the basis information.

### [Supplementary Note 8]

The action recommendation device according to any one of Supplementary Notes 5 to 7, further comprising
a determination means configured to determine whether or not a timing of recommending an action to the target person comes up,
wherein upon determining that the timing has come up, the output means is configured to output the information regarding the recommended action.

### [Supplementary Note 9]

The action recommendation device according to any one of Supplementary Notes 5 to 8, further comprising
a target person data acquisition means configured to acquire target person data which is data regarding the target person,
wherein the history information acquisition means is configured to generate the history information based on the target person data.

### [Supplementary Note 10]

The action recommendation device according to Supplementary Note 9,
wherein the target person data includes a signal outputted by a sensor which measures the target person.

### [Supplementary Note 11]

The action recommendation device according to Supplementary Note 9 or 10,
wherein the target person data acquisition means is configured to acquire the target person data from a terminal device used by the target person,
wherein the output control means is configured to transmit the information regarding the recommended action to the terminal device.

### [Supplementary Note 12]

The action recommendation device according to any one of Supplementary Notes 9 to 11, wherein the history information acquisition means is configured to generate the history information based on the diagnosis data of a medical checkup undergone by the target person.

### [Supplementary Note 13]

A learning method executed by a computer, the learning method comprising:
acquiring history information indicating a history of a health state of a target person and an action of the target person contributing to variation in the health state, and success/failure information indicating whether or not the action contributed to the variation in the health state of the target person; and
training a model based on the history information and the success/failure information, wherein the model is configured to output information regarding a recommended action recommended to improve the health state of the target person upon receiving an input of the history information indicating the history of the action and the health state of the target person.

### [Supplementary Note 14]

An action recommendation method executed by a computer, the recommendation method including:
acquiring history information indicating a history of a health state of a target person and an action of the target person contributing to variation in the health state;
determining a recommended action to be recommended to the target person based on the history information and a recommendation model; and
outputting information regarding the recommended action,
wherein the recommendation model is a model which learned a relation between each health state of subjects and each recommended action to be recommended to improve the each health state of the subjects, based on history information indicating histories of health states of the subjects and actions of the subjects which contribute to variation in the health states.

### [Supplementary Note 15]

A storage medium storing a program executed by a computer, the program causing the computer to:
acquire history information indicating a history of a health state of a target person and an action of the target person contributing to variation in the health state, and success/failure information indicating whether or not the action contributed to the variation in the health state of the target person; and
train a model based on the history information and the success/failure information, wherein the model is configured to output information regarding a recommended action recommended to improve the health state of the target person upon receiving an input of the history information indicating the history of the action and the health state of the target person.

### [Supplementary Note 16]

A storage medium storing a program executed by a computer, the program causing the computer to:
acquiring history information indicating a history of a health state of a target person and an action of the target person contributing to variation in the health state;
determining a recommended action to be recommended to the target person based on the history information and a recommendation model; and
outputting information regarding the recommended action,
wherein the recommendation model is a model which learned a relation between each health state of subjects and each recommended action to be recommended to improve the each health state of the subjects, based on history information indicating histories of health states of the subjects and actions of the subjects which contribute to variation in the health states.

### [Supplementary Note 17]

A learning system comprising:
an acquisition means configured to acquire history information indicating a history of a health state of a target person and an action of the target person contributing to variation in the health state, and success/failure information indicating whether or not the action contributed to the variation in the health state of the target person; and
a learning means configured to train a model based on the history information and the success/failure information, wherein the model is configured to output information regarding a recommended action recommended to improve the health state of the target person upon receiving an input of the history information indicating the history of the action and the health state of the target person.

### [Supplementary Note 18]

The learning system according to Supplementary Note 17,
wherein the history information is information that indicates the action and the health state observed after the action alternately in time series.

### [Supplementary Note 19]

The learning system according to Supplementary Note 17 or 18,
wherein the history information includes, as a record of the action, information regarding a type of the action and a degree of the action.

### [Supplementary Note 20]

The learning system according to any one of Supplementary Notes 17 to 19,
wherein the history information includes, as a record of the health state, an indicator related to the health state used for calculation of a benchmark indicator used as a criterion for determining whether or not the history is the success example.

### [Supplementary Note 21]

An action recommendation system comprising:
a history information acquisition means configured to acquire history information indicating a history of a health state of a target person and an action of the target person contributing to variation in the health state;
a recommended action determination means configured to determine a recommended action to be recommended to the target person based on the history information and a recommendation model; and
an output means configured to output information regarding the recommended action,
wherein the recommendation model is a model which learned a relation between each health state of subjects and each recommended action to be recommended to improve the each health state of the subjects, based on history information indicating histories of health states of the subjects and actions of the subjects which contribute to variation in the health states.

### [Supplementary Note 22]

The action recommendation system according to Supplementary Note 21,
wherein the recommended action determination means is configured to generate recommended action promotion information for notifying the target person of the recommended action, and
wherein the output means is configured to further output the recommended action promotion information.

### [Supplementary Note 23]

The action recommendation system according to Supplementary Note 21 or 22, further comprising
a basis information generation means configured to generate basis information regarding a basis of determining the recommended action,
wherein the output means is configured to output the basis information.

### [Supplementary Note 24]

The action recommendation system according to any one of Supplementary Notes 21 to 23, further comprising
a determination means configured to determine whether or not a timing of recommending an action to the target person comes up,
wherein upon determining that the timing has come up, the output means is configured to output the information regarding the recommended action.

### [Supplementary Note 25]

The action recommendation system according to any one of Supplementary Notes 21 to 24, further comprising
a target person data acquisition means configured to acquire target person data which is data regarding the target person,
wherein the history information acquisition means is configured to generate the history information based on the target person data.

### [Supplementary Note 26]

The action recommendation system according to Supplementary Note 25,
wherein the target person data includes a signal outputted by a sensor which measures the target person.

### [Supplementary Note 27]

The action recommendation system according to Supplementary Note 25 or 26,
wherein the target person data acquisition means is configured to acquire the target person data from a terminal device used by the target person,
wherein the output control means is configured to transmit the information regarding the recommended action to the terminal device.

### [Supplementary Note 28]

The action recommendation system according to any one of Supplementary Notes 25 to 27,
wherein the history information acquisition means is configured to generate the history information based on the diagnosis data of a medical checkup undergone by the target person.

While the invention has been particularly shown and described with reference to example embodiments thereof, the invention is not limited to these example embodiments. It will be understood by those of ordinary skilled in the art that various changes in form and details may be made therein without departing from the spirit and scope of the present invention as defined by the claims. In other words, it is needless to say that the present invention includes various modifications that could be made by a person skilled in the art according to the entire disclosure including the scope of the claims, and the technical philosophy. All Patent and Non-Patent Literatures mentioned in this specification are incorporated by reference in its entirety.

### INDUSTRIAL APPLICABILITY

It can be used for services related to health management (including self-management) such as diet support, health promotion, health management of athletes, and management of patient's rehabilitation.

DESCRIPTION OF REFERENCE NUMERALS

| | |
|---|---|
| 1, 1X | Learning device |
| 2. 2A, 2X | Action recommendation device |
| 3 | Storage device |
| 4 | Input device |
| 5 | Output device |
| 6 | Sensor |
| 8 | Terminal device |
| 100, 100A | Action recommendation device |

What is claimed is:

1. A learning device comprising:
at least one memory configured to store instructions; and
at least one processor configured to execute the instructions to:
acquire history information indicating a history of a health state of a target person and an action of the target person contributing to variation in the health state, and success/failure information indicating whether or not the action contributed to the variation in the health state of the target person; and
train a model based on the history information and the success/failure information, wherein the model is configured to output information regarding a recommended action recommended to improve the health state of the target person upon receiving an input of the history information indicating the history of the action and the health state of the target person.

2. The learning device according to claim 1,
wherein the history information is information that indicates the action and the health state observed after the action alternately in time series.

3. The learning device according to claim 1,
wherein the history information includes, as a record of the action, information regarding a type of the action and a degree of the action.

4. The learning device according to claim 1,
wherein the history information includes, as a record of the health state, an indicator related to the health state used for calculation of a benchmark indicator used as a criterion for determining whether or not the history is the success example.

5. An action recommendation device comprising:
at least one memory configured to store instructions; and
at least one processor configured to execute the instructions to:
acquire history information indicating a history of a health state of a target person and an action of the target person contributing to variation in the health state;
determine a recommended action to be recommended to the target person based on the history information and a recommendation model; and
output information regarding the recommended action,
wherein the recommendation model is a model which learned a relation between each health state of subjects and each recommended action to be recommended to improve the each health state of the subjects, based on history information indicating histories of health states of the subjects and actions of the subjects which contribute to variation in the health states.

6. The action recommendation device according to claim 5,
wherein the at least one processor is configured to execute the instructions to generate recommended action promotion information for notifying the target person of the recommended action, and
wherein the at least one processor is configured to execute the instructions to further output the recommended action promotion information.

7. The action recommendation device according to claim 5,
wherein the at least one processor is configured to further execute the instructions to generate basis information regarding a basis of determining the recommended action,
wherein the at least one processor is configured to execute the instructions to output the basis information.

8. The action recommendation device according to claim 5,
wherein the at least one processor is configured to execute the instructions to determine whether or not a timing of recommending an action to the target person comes up,
wherein upon determining that the timing has come up, the at least one processor is configured to execute the instructions to output the information regarding the recommended action.

9. The action recommendation device according to claim 5,
wherein the at least one processor is configured to execute the instructions to acquire target person data which is data regarding the target person,
wherein the at least one processor is configured to execute the instructions to generate the history information based on the target person data.

10. The action recommendation device according to claim 9,
wherein the target person data includes a signal outputted by a sensor which measures the target person.

11. The action recommendation device according to claim 9,
wherein the at least one processor is configured to execute the instructions to acquire the target person data from a terminal device used by the target person,
wherein the at least one processor is configured to execute the instructions to transmit the information regarding the recommended action to the terminal device.

12. The action recommendation device according to claim 9,
wherein the at least one processor is configured to execute the instructions to generate the history information based on the diagnosis data of a medical checkup undergone by the target person.

13. A learning method executed by a computer, the learning method comprising:
acquiring history information indicating a history of a health state of a target person and an action of the target person contributing to variation in the health state, and success/failure information indicating whether or not the action contributed to the variation in the health state of the target person; and
training a model based on the history information and the success/failure information, wherein the model is configured to output information regarding a recommended action recommended to improve the health state of the target person upon receiving an input of the history information indicating the history of the action and the health state of the target person.

14-16. (canceled)
